(19) United States
(12) Patent Application Publication
(10) Pub. No.: US 2019/0164661 A1
(43) Pub. Date: May 30, 2019

(54) WATER-BASED VEHICLE FOR ELECTROCONDUCTIVE PASTE

(71) Applicant: Heraeus Precious Metals North America Conshohocken LLC, West Conshohocken, PA (US)

(72) Inventor: LIXIN SONG, King of Prussia, PA (US)

(21) Appl. No.: 15/822,413

(22) Filed: Nov. 27, 2017

(51) Int. Cl. H01B 1/16; H01L 31/0224; C08L 29/04; C08L 39/06; C08L 71/02; C08K 5/00; C08K 5/053; C07C 31/20; C03C 8/18

(52) U.S. Cl. CPC H01B 1/16; H01L 31/022425; C08L 29/04; C08L 39/06; C03C 8/18; C08K 5/005; C08K 5/053; C07C 31/202; C08L 71/02

(57) ABSTRACT

The invention relates to a water-based vehicle used in the manufacture of an electroconductive silver paste. The water-based vehicle comprises a binder, a stabilizer, and water. The preferred embodiment of the invention utilizes at least one of polyvinylpyrrolidone, polyvinyl alcohol, and polyethelene glycol as the binder; and ethylene glycol as the stabilizer. Another aspect of the invention relates to an electroconductive paste composition based on the water-based vehicle. The preferred embodiment utilizes a metallic particle, a glass frit, and a water-based vehicle comprising a binder, a stabilizer, and water. The electroconductive paste of a high metallic content exhibits excellent storage stability.

### FIELD OF THE INVENTION

This invention relates to an electroconductive paste as utilized in solar panel technology. Specifically, in one aspect, the invention relates to a water-based vehicle used in the formulation of an electroconductive paste. Another aspect of the invention relates to an electroconductive paste composition. Another aspect of the invention relates to a solar cell produced by applying an electroconductive paste comprised of metallic particles, glass frit, and an aqueous vehicle.

### BACKGROUND OF THE INVENTION

Solar cells are devices that convert the energy of light into electricity using the photovoltaic effect. Solar power is an attractive green energy source because it is sustainable and produces only non-polluting by-products. Accordingly, a great deal of research is currently being devoted to developing solar cells with enhanced efficiency while continuously lowering material and manufacturing costs. When light hits a solar cell, a fraction of the incident light is reflected by the surface and the remainder is transmitted into the solar cell. The photons of the transmitted light are absorbed by the solar cell, which is usually made of a semiconducting material such as silicon. The energy from the absorbed photons excites electrons of the semiconducting material from their atoms, generating electron-hole pairs. These electron-hole pairs are then separated by p-n junctions and collected by conductive electrodes which are applied on the solar cell surface.

The most common solar cells are those based on silicon, more particularly, a p-n junction made from silicon by applying an n-type diffusion layer onto a p-type silicon substrate, coupled with two electrical contact layers or electrodes. In a p-type semiconductor, dopant atoms are added to the semiconductor in order to increase the number of free charge carriers (positive holes). In the case of silicon, a trivalent atom is substituted into the crystal lattice. Essentially, the doping material takes away weakly bound outer electrons from the semiconductor atoms. One example of a p-type semiconductor is silicon with a boron or aluminum dopant. Solar cells can also be made from n-type semiconductors. In an n-type semiconductor, the dopant atoms provide extra electrons to the host substrate, creating an excess of negative electron charge carriers. Such doping atoms usually have one more valence electron than one type of the host atoms. The most common example is atomic substitution in group IV solids (silicon, germanium, tin), which contain four valence electrons, by group V elements (phosphorus, arsenic, antimony), which contain five loosely bound valence electrons. One example of an n-type semiconductor is silicon with a phosphorous dopant.

In order to minimize reflection of the sunlight by the solar cell, an antireflection coating (ARC), such as silicon nitride, silicon oxide, alumina oxide or titanium oxide, is applied to the n-type or p-type diffusion layer to increase the amount of light coupled into the solar cell. The ARC is typically non-conductive and may also passivate the surface of the silicon substrate.

Silicon solar cells typically have electroconductive pastes applied to both their front and back surfaces. As part of the metallization process, a rear contact is typically first applied to the silicon substrate, such as by screen printing a back side silver paste or silver/aluminum paste to form soldering pads. Next, an aluminum paste is applied to the entire back side of the substrate to form a back surface field (BSF), and the cell is then dried. Next, using a different type of electroconductive paste, a metal contact may be screen printed onto the front side antireflection layer to serve as a front electrode. This electrical contact layer on the face or front of the cell, where light enters, is typically present in a grid pattern made of “finger lines” and “bus bars,” rather than a complete layer, because the metal grid materials are typically not transparent to light. The silicon substrate with printed front side and back side paste is then fired at a temperature of approximately 700-975° C. After firing, the front side paste etches through the antireflection layer, forms electrical contact between the metal grid and the semiconductor, and converts the metal pastes to metal electrodes. On the back side, the aluminum diffuses into the silicon substrate, acting as a dopant which creates the BSF. The resulting metallic electrodes allow electricity to flow to and from solar cells connected in a solar panel.

A description of a typical solar cell and the fabrication method thereof may be found, for example, in European Patent Application Publication No. 1713093.

To assemble a panel, multiple solar cells are connected in series and/or in parallel and the ends of the electrodes of the first cell and the last cell are preferably connected to output wiring. The solar cells are typically encapsulated in a transparent thermal plastic resin, such as silicon rubber or ethylene vinyl acetate. A transparent sheet of glass is placed on the front surface of the encapsulating transparent thermal plastic resin. A back protecting material, for example, a sheet of polyethylene terephthalate coated with a film of polyvinyl fluoride having good mechanical properties and good weather resistance, is placed under the encapsulating thermal plastic resin. These layered materials may be heated in an appropriate vacuum furnace to remove air, and then integrated into one body by heating and pressing. Furthermore, since solar cells are typically left in the open air for a long time, it is desirable to cover the circumference of the solar cell with a frame material consisting of aluminum or the like.

A typical electroconductive paste contains metallic particles, glass frit (glass particles), and a vehicle. These components must be carefully selected to take full advantage of the potential of the resulting solar cell. For example, it is necessary to maximize the contact between the metallic paste and silicon surface, and the metallic particles themselves, so that the charge carriers can flow through the interface and finger lines to the bus bars. Silver is typically the metal of choice for the electroconductive paste. The glass particles in the composition etch through the antireflection coating layer, helping to build contacts between the metal and the n+ type Si. On the other hand, the glass must not be so aggressive that it shunts the p-n junction after firing. Thus, minimizing contact resistance is desired with the p-n junction kept intact so as to achieve improved efficiency. Known compositions have high contact resistance due to the insulating effect of the glass in the interface of the metallic layer and silicon wafer, as well as other disadvantages such as high recombination in the contact area. Further, the composition of the organic vehicle can affect the potential of the resulting solar cell as well.

All current silver paste for solar applications on the market are in an organic vehicle. The organic solvents allow wettabililty of the cell and solid paste ingredients, control of rheological and printing behavior, and a slow evaporation to ensure a smooth screen printing process. However the organic system usually have a negative impact on the environment and the operator's health.

Accordingly, there is a need for a water-based vehicle for an electroconductive paste whose performance is comparable to the traditional organic-based electroconductive pastes.

US 2008/0193667 relates to an ink jet printable composition of nano metal particles in a liquid carrier. The '667 publication further discloses that useful liquid carriers include water, organic solvents, and combinations thereof.

US 2011/0151614 is directed to a process for producing electrodes for solar cells by irradiating a dispersion which includes electrically conductive particles, glass frit, a matrix material, and a solvent. Water and mixtures of two or more organic solvents are examples of a solvent for the dispersion.

WO 2011/038657 concerns a conductive paste comprising a conductive metal powder, an inorganic adhesive, an aqueous adhesive including a water-soluble polymer, and an additive.

### SUMMARY OF THE INVENTION

The invention provides a water-based vehicle for use in an electroconductive paste comprising: 1) a binder; 2) a stabilizer; and 3) water. According to another embodiment, the water-based vehicle further comprises a thixatropic agent.

According to the invention, the binder comprises at least one of polynylpyrrolidone (PVP), polyvinyl alcohol (PVA), polyethylene glycol (PEG), cellulose ethers, polyethylene oxide, aqueous polyurethane resin, polyvinylbutylate (PVB), and combinations thereof.

According to the invention, the binder is from about 5 to about 30 wt %, from about 10 to about 20 wt %, or from about 10 to about 15 wt %, of the water-based vehicle.

According to the invention, the stabilizer comprises a glycol such as ethylene glycol, propylene glycol, 1,4-butane diol, and diethylene glycol. The stabilizer is from about 0 to about 30 wt %, from about 3 to about 30 wt %, from about 5 to about 15 wt %, or from about 5 to about 10 wt % of the water-based vehicle.

According to the invention, water in the water-based vehicle is above 50% wt %, from about 60 to about 90 wt %, from about 70 to about 80 wt %, or about 80% of the water-based vehicle.

According to the invention, the weight ratio of water to stabilizer is from about 3 to about 30, from about 5 to about 20, from about 7 to about 18, or from about 8 to about 16. In a preferred embodiment, the weight ratio of water to ethylene glycol is from about 5 to about 20, from about 7 to about 18, or from about 8 to about 16.

The invention also provides an electroconductive paste for use in solar cell technology comprising a metallic particle, a glass frit, and a water-based vehicle comprising 1) a binder, 2) a stabilizer, and 3) water.

According to another aspect of the invention, the metallic particles are at least one of silver, gold, copper, and nickel, preferably silver. According to one aspect of the invention, the metallic particles are from about 60 to about 95 wt %, from about 70 to about 90 wt %, from about 80 to about 90 wt %, or about 88 wt % of the paste.

According to another aspect of the invention, the glass frit is from about 1 to about 10 wt %, from about 2 to about 8 wt %, from about 2 to about 5 wt %, or about 2 wt % of the paste.

According to a further aspect of the invention, the water-based vehicle is from about 1 to about 20 wt %, from about 5 to about 15 wt %, about 8 wt %, about 8 wt %, about 10 wt %, or about 11 wt % of the paste.

According to another aspect of the invention, the water-based vehicle or the paste further comprises a thixatropic agent. The optional thixatropic agent may be added during the preparation of the electroconductive paste rather than during the preparation of the water-based vehicle. The thixatropic agent is from about 5 to about 15 wt %, from about 7 to about 12 wt %, or about 10 wt % based on the weight of the water-based vehicle. Alternatively, the thixatropic agent is from about 0.1 to about 5 wt %, from about 0.5 to about 2 wt %, or about 1 wt % based on the weight of the paste.

The invention further provides a solar cell produced by applying an electroconductive paste of the invention to a silicon wafer, and firing the silicon wafer.

### DETAILED DESCRIPTION

The invention relates to a water-based vehicle used in an electroconductive paste. The electroconductive paste composition comprises: metallic particles, a glass frit, and a water-based vehicle. The water-based vehicle of the invention is particularly useful for a paste with high metallic particle contents. While not limited to such an application, such a paste may be used to form an electrical contact layer or electrode in a solar cell. Specifically, the paste may be applied to the front side or to the back side of a solar cell.

### Water-Based Vehicle

One aspect of the invention relates to the composition of the water-based vehicle for the electroconductive paste. A desired vehicle is one which is low in viscosity, allowing for fine line printability, and has optimal stability when combined with metallic particles. According to the invention, the water-based vehicle comprises a binder, a stabilizer, and water. The water-based vehicle may also comprise a thixatropic agent.

According to one aspect, the binder comprises at least one water-soluble polymer. Examples include polyvinylpyrrolidone (PVP), polyvinyl alcohol (PVA), polyethylene glycol (PEG), cellulose ethers, polyethylene oxide, aqueous polyurethane resin, polyvinylbutylate (PVB), and combinations thereof. The binder is from about 5 to about 30 wt %, from about 10 to about 20 wt %, or from about 10 to about 15 wt % of the water-based vehicle. In a preferred embodiment, the binder is from about 10 to about 15 wt % of the water-based vehicle. In another preferred embodiment, the binder is polyvinyl pyrrolidone (PVP). Preferably the PVP has a molecular weight from about 5 K to about 5,000 K Dalton, form about 20 K Dalton to about 500,000 K Dalton, or from about 30 K Dalton to about 400 K Dalton. In another preferred embodiment, the PVP is PVP-K30 having a molecular weight of about 40 K Dalton. In another preferred embodiment, the PVP is PVP-K90 having a molecular weight of about 360 K Dalton. In another preferred embodiment, PVA is the binder. The binder, especially PVP and PVA, enhances the paste's ability to be dispersed uniformly, and is highly compatible with silver particles.

According to another aspect, a stabilizer (surfactant) is used in the water-based vehicle from 0 to about 30 wt % of the water-based vehicle. Examples of a stabilizer include glycols, such as ethylene glyclol, propylene glycol, 1,4-butane diol and diethylene glycol. In a preferred embodiment, ethylene glycol is used. In another preferred embodiment, the stabilizer is from about 3 to about 30 wt %, from about 5 to about 15 wt %, or from about 5 to about 10 wt % of the water-based vehicle.

According to a further aspect, water in the water-based vehicle is above 50 wt %, from about 60 to about 90 wt %, or from about 70 to about 80 wt % of the water-based vehicle. In another preferred embodiment, water is about 80 wt % of the water-based vehicle.

In one embodiment, the weight ratio of water to the stabilizer is from about 3 to about 30, from about 5 to about 20, from about 7 to about 18, or from about 8 to about 16. In a preferred embodiment, the weight ratio of water to ethylene glycol is from about 5 to about 20, from about 7 to about 18, or from about 8 to about 16.

According to an additional aspect, the water-based vehicle may further comprise a thixatropic agent. Any thixatropic agents familiar to one having ordinary skill in the art may be used with the water-based vehicle of the invention. For example, without limitation, thixatropic agents may be derived from natural origin, e.g., castor oil, or they may be synthesized. Commercially available thixatropic agents can also be used with the invention. The thixatropic agent is from about 5 to about 15 wt %, preferably from about 7 to about 12 wt %, preferably about 10 wt %, of the water-based vehicle. The thixatropic agent can be added to the vehicle or incorporated during the paste preparation. Thus, alternatively the thixatropic agent is from about 0.1 to about 5 wt %, from about 0.5 to about 2 wt %, or about 1 wt % based on the weight of the paste.

The water-based vehicle according to the preferred embodiment typically exhibits a viscosity range of 140-200 kcPs, and a thixatropic index (viscosity at 1 rpm/10 rpm, Brookfield method) of 5-10.

In one embodiment, the water-based vehicle comprises from about 5 to about 30 wt % of a binder, from about 3 to about 30 wt % of a stabilizer, and above 50 wt % of water. In one embodiment, the water-based vehicle comprises from about 5 to about 30 wt %, from 10 to about 20 wt %, or from about 10 to about 15 wt % of PVP as the binder. In another embodiment, the water-based vehicle comprises from about 3 to about 30 wt %, from about 5 to about 10 wt %, or from about 5 to about 10 wt % of ethylene glycol as a stabilizer. In another embodiment, the water-based vehicle comprises from about 60 to about 90 wt %, from about 70 to about 80 wt %, or about 80 wt % of water.

In yet another embodiment, the water-based vehicle comprises from about 5 to about 30 wt % PVA as the binder, from about 3 to about 20 wt % ethylene glycol as the stabilizer, and from about 60 to about 90 wt % water. In another embodiment, the water-based vehicle comprises from about 10 to about 20 wt % PVA as the binder, from about 5 to about 10 wt % ethylene glycol as the stabilizer, and about 80 wt % water.

One aspect of the present invention relates to the composition of an electroconductive paste used to form the front side or the backside of a solar cell. The electroconductive paste composition according to the present invention is comprised of metallic particles, a glass frit, and a water-based vehicle. The electroconductive paste composition may further comprise a thixatropic agent incorporated into the water-based vehicle or into the paste.

The preferred water-based vehicles in the context of the invention are emulsions or dispersions. The preferred water-based vehicles are those which provide optimal stability of constituents within the electroconductive paste and endow the electroconductive paste with a certain viscosity to optimize printability.

### Metallic Particles

The metallic particles known in the art suitable for uses as solar cell surface electrodes that are also easy to solder, and mixtures or alloys thereof, can be used with the present invention. In one embodiment, the metallic particles are at least one of silver, aluminum, gold and nickel, or any alloys thereof. The metallic particles are typically from about 60 to about 95 wt % of the paste composition. In another embodiment, the metallic particles are from about 70 to about 90 wt %. In another embodiment, the metallic particles are from about 80 to about 90 wt %. In another embodiment, the metallic particles are about 88 wt %. In a preferred embodiment, the metallic particles are silver. In another embodiment, the conductive particles are a mixture of silver and aluminum or alloy.

The conductive particles may be present as elemental metal, one or more metal derivatives, or a mixture thereof. Suitable silver derivatives include, for example, silver alloys and/or silver salts, such as silver halides (e.g., silver chloride), silver nitrate, silver acetate, silver trifluoroacetate, silver orthophosphate, and combinations thereof.

The conductive particles can exhibit a variety of shapes, surfaces, sizes, surface area to volume ratios, oxygen content and oxide layers. A large number of shapes are known in the art. Some examples are spherical, angular, elongated (rod or needle like) and flat (sheet like). Conductive metallic particles may also be present as a combination of particles of different shapes. Metallic particles with a shape, or combination of shapes, which favors packaging are preferred according to the invention. One way to characterize such shapes without considering the surface nature of the particles is through the following parameters: length, width and thickness. In the context of the invention, the length of a particle is given by the length of the longest spatial displacement vector, both endpoints of which are contained within the particle. The width of a particle is given by the length of the longest spatial displacement vector perpendicular to the length vector defined above both endpoints of which are contained within the particle. The thickness of a particle is given by the length of the longest spatial displacement vector perpendicular to both the length vector and the width vector, both defined above, both endpoints of which are contained within the particle.

In one embodiment according to the invention, metallic particles with shapes as uniform as possible are preferred (i.e. shapes in which the ratios relating the length, the width and the thickness are as close as possible to 1, preferably all ratios lying in a range from about 0.7 to about 1.5, more preferably in a range from about 0.8 to about 1.3 and most preferably in a range from about 0.9 to about 1.2). Examples of preferred shapes for the metallic particles in this embodiment are spheres and cubes, or combinations thereof, or combinations of one or more thereof with other shapes. In another embodiment according to the invention, metallic particles are preferred which have a shape of low uniformity, preferably with at least one of the ratios relating the dimensions of length, width and thickness being above about 1.5, more preferably above about 3 and most preferably above about 5. Preferred shapes according to this embodiment are flake shaped, rod or needle shaped, or a combination of flake shaped, rod or needle shaped with other shapes.

The particle diameter d₅₀ and the associated values, d₁₀ and d₉₀, are characteristics of particles well known to the person skilled in the art. It is preferred according to the invention that the median particle diameter d₅₀ of the metallic particles lie in a range from about 1 to about 2 μm. The determination of the particle diameter d₅₀ is well known to a person skilled in the art.

In one embodiment of the invention, the metallic particles have a d₁₀ about 0.9-1.2 μm, and a d₉₀ about 2.4-3 μm.

The metallic particles may be present with a surface coating. Any such coating known in the art, and which is considered to be suitable in the context of the invention, may be employed on the metallic particles. Preferred coatings according to the invention are those coatings which promote adhesion and wetting characteristics of the metal particles. If such a coating is present, it is preferred according to the invention for that coating to correspond to no more than about 10 wt %, preferably no more than about 8 wt %, most preferably no more than about 5 wt %, in each case based on the total weight of the metallic particles.

### Glass Frit

In a preferred embodiment, the glass frit may be from about 1 to about 10 wt %, from about 2 to about 8 wt %, or from about 2 to about 5 wt % of the paste composition. In another preferred embodiment, the glass frit is about 2 wt %.

The glass frits are not particularly limited. Pb-based glass frits, for example, PbO—B₂O₃—SiO₂-based glass frits, PbO—TeO—ZnO—WO₃-based glass frits, and the like; Pb-free glass frits, for example, Bi₂O₃—B₂O₃—SiO₂—CeO₂—LiO₂—NaO₂-based glass frits, and the like may be used. Preferably, the glass frit may contain Pb and/or Te with Zn, W, Si, Al, alkaline oxide and alkaline earth metal oxide. The glass frit may also be Pb or Te free. The shape and size of the glass frits are not particularly limited, and those known in the art can be used. As for the shape of the glass frits, a spherical shape, an amorphous shape and the like may be mentioned. The average particle dimension of the glass first may be 0.01 to 10 μm, and preferably 0.05 to 1 μm, from the viewpoint of workability or the like. The average particle dimension is as previously described above, but in the case of an amorphous shape, the dimension refers to the average of the longest diameter.

### Forming the Electroconductive Paste

The electroconductive paste composition may be prepared by any method for preparing a paste composition known in the art. As an example, without limitation, the paste components may be mixed, such as with a mixer, then passed through a three roll mill, for example, to make a dispersed uniform paste. The electroconductive paste of the current invention comprises a metallic particle, a glass frit, and a water-based vehicle.

The metallic particle is typically from about 60 to about 95 wt % of the paste composition. In another embodiment, the metallic particles are from about 70 to about 90 wt %. In another embodiment, the metallic particles are from about 80 to about 90 wt %. In another embodiment, the metallic particles are about 88 wt %.

The glass frit may be from about 1 to about 10 wt %, from about 2 to about 8 wt %, or from about 2 to about 5 wt % of the paste composition. In another preferred embodiment, the glass frit is about 2 wt %.

The water-based vehicle is from about 1 to about 20 wt % of the paste. Preferably, the water-based vehicle is from about 5 to about 15 wt % of the paste. More preferably, the water-based vehicle is about 8 wt %, about 9 wt %, about 10 wt %, or about 11 wt % of the paste.

The optional thixatropic agent may be added during the preparation of the electroconductive paste rather than during the preparation of the water-based vehicle. The thixatropic agent is from about 5 to about 15 wt %, from about 7 to about 12 wt %, or about 10 wt % of the water-based vehicle. The thixatropic agent can be added to the vehicle or incorporated during the paste preparation. Thus, alternatively the thixatropic agent is from about 0.1 to about 5 wt %, from about 0.5 to about 2 wt %, or about 1 wt % based on the weight of the paste.

Upon storage at 25° C., the electroconductive paste of the current invention remains stable over a period of at least one month. The paste of the current invention exhibits excellent storage stability.

### Method of Preparing a Solar Cell

A solar cell may be prepared by applying the electroconductive paste of the invention to an antireflection coating, such as silicon nitride, silicon oxide, titanium oxide or aluminum oxide, on the front side of a semiconductor substrate, such as a silicon wafer. A backside electroconductive paste is then applied to the backside of the solar cell to form soldering pads. An aluminum paste is then applied to the backside of the substrate, overlapping the edges of the soldering pads formed from the backside electroconductive paste, to form the BSF.

The electroconductive pastes may be applied in any manner known in the art and considered suitable in the context of the invention. Examples include, but are not limited to, impregnation, dipping, pouring, dripping on, injection, spraying, knife coating, curtain coating, brushing or printing or a combination of at least two thereof. Preferred printing techniques are ink-jet printing, screen printing, tampon printing, offset printing, relief printing or stencil printing or a combination of at least two thereof. It is preferred according to the invention that the electroconductive paste is applied by printing, preferably by screen printing. Specifically, the screens preferably have mesh opening with a diameter of about 40 μm or less (e.g., about 35 μm or less, about 30 μm or less). At the same time, the screens preferably have a mesh opening with a diameter of at least 10 μm.

The substrate is then subjected to one or more thermal treatment steps, such as, for example, conventional over drying, infrared or ultraviolet curing, and/or firing. In one embodiment the substrate may be fired according to an appropriate profile. Firing sinters the printed electroconductive paste so as to form solid electrodes. Firing is well known in the art and can be effected in any manner considered suitable in the context of the invention. It is preferred that firing be carried out above the T_{g} of the glass frit materials.

According to the invention, the maximum temperature set for firing is below about 900° C., preferably below about 860° C. Firing temperatures as low as about 800° C. have been employed for obtaining solar cells. Firing temperatures should also allow for effective sintering of the metallic particles to be achieved. The firing temperature profile is typically set so as to enable the burnout of organic materials from the electroconductive paste composition. The firing step is typically carried out in air or in an oxygen-containing atmosphere in a belt furnace. It is preferred for firing to be carried out in a fast firing process with a total firing time of at least 30 seconds, and preferably at least 40 seconds. At the same time, the firing time is preferably no more than about 3 minutes, more preferably no more than about 2 minutes, and most preferably no more than about 1 minute. The time above 600° C. is most preferably in a range from about 3 to 7 seconds. The substrate may reach a peak temperature in the range of about 700 to 900° C. for a period of about 1 to 5 seconds. The firing may also be conducted at high transport rates, for example, about 100-700 cm/min, with resulting hold-up times of about 0.5 to 3 minutes. Multiple temperature zones, for example 3-12 zones, can be used to control the desired thermal profile.

Firing of electroconductive pastes on the front and back faces can be carried out simultaneously or sequentially. Simultaneous firing is appropriate if the electroconductive pastes applied to both faces have similar, preferably identical, optimum firing conditions. Where appropriate, it is preferred according to the invention for firing to be carried out simultaneously. Where firing is carried out sequentially, it is preferable according to the invention for the back electroconductive paste to be applied and fired first, followed by application and firing of the electroconductive paste to the front face of the substrate.

### Measuring Properties of Electroconductive Paste

The electrical performance of a solar cell is measured using a commercial IV-tester “cetisPV-CTL1” from Halm Elektronik GmbH. All parts of the measurement equipment as well as the solar cell to be tested are maintained at 25° C. during electrical measurement. This temperature should be measured simultaneously on the cell surface during the actual measurement by a temperature probe. The Xe Arc lamp simulates the sunlight with a known AM1.5 intensity of 1000 W/m² on the cell surface. To bring the simulator to this intensity, the lamp is flashed several times within a short period of time until it reaches a stable level monitored by the “PVCTControl 4.313.0” software of the IV-tester. The Halm IV tester uses a multi-point contact method to measure current (I) and voltage (V) to determine the solar cell's IV-curve. To do so, the solar cell is placed between the multi-point contact probes in such a way that the probe fingers are in contact with the bus bars (i.e., printed lines) of the solar cell. The numbers of contact probe lines are adjusted to the number of bus bars on the cell surface. All electrical values were determined directly from this curve automatically by the implemented software package. As a reference standard, a calibrated solar cell from ISE Freiburg consisting of the same area dimensions, same wafer material, and processed using the same front side layout, was tested and the data was compared to the certificated values. At least five wafers processed in the very same way were measured and the data was interpreted by calculating the average of each value. The software PVCTControl 4.313.0 provided values for efficiency, fill factor, short circuit current, series resistance and open circuit voltage.

The present invention is illustrated by, but not limited to, the following examples. Many modifications and variations will be apparent to those of ordinary skill in the art.

### Example 1

The aqueous vehicle was prepared by combining water, PVP-K30, and ethylene glycol. The mixture was heated to a temperature of 80° C. while stirring, and then maintained for a total of 30 minutes. The aqueous vehicle was then cooled to room temperature. The aqueous vehicle was then mixed with silver particles, a glass frit and a thixotrope [amide based] using a speed mixer. The resulting paste was screen printed onto a solar wafer at a speed of 150 mm/s, using screen 325 (mesh)*0.9 (mil, wire diameter)*0.6 (mil, emulsion thickness)*50 μm (finger line opening) (Calendar screen), and the printed wafer was then fired at the appropriate profile.

Aqueous vehicle 1 (V1) and aqueous vehicle 2 (V2) were prepared according to Table 1. The vehicle used in the control includes the following ingredients: carbitol (solvent), diaminopropane-ditallates (surfactant), ethyl cellulose (binder) and a thixotrope [amide based].

TABLE 1

| | V1 (wt %) | V2 (wt %) | V3 (wt %) | Control |
|---|---|---|---|---|
| Water | 80 | 80 | 80 | Commercial |
| | | | | SOL 9621 |
| Ethylene glycol | 10 | 5 | 0 | |
| PVP-K30 | 10 | 15 | 20 | |
| Printability | + | + | − | + |

V1 and V2 were formulated into paste 1 (P1) and paste 2 (P2) respectively according to Table 2.

TABLE 2

| | P1 (wt %) | P2 (wt %) | Control |
|---|---|---|---|
| Ag powder | 88 | 88 | Commercial |
| | | | SOL 9621 |
| Glass frit | 2 | 2 | |
| V1 | 9 | | |
| V2 | | 9 | |
| Thixatrope | 1 | 1 | 1 |

### Example 2

V1 and V2 were each converted to a paste in accordance with the composition provided in Table 2. Solar cells were prepared by using either the fresh pastes or the pastes after storing at 25° C. for one month. The electrical performance of the solar cells was measured. The solar cell efficiency prepared from the pastes before or after storage is compared in Table 3 below. Before/after storage: 0=no changes, −=negative impact.

TABLE 3

| | | | Control |
|---|---|---|---|
| | | | Commercial |
| Vehicle in Paste | V1 | V2 | SOL 9621 |
| Solar Cell | 0 | 0 | − |
| Efficiency | | | |

### Example 3

The solar cells produced using the exemplary electroconductive pastes P1 and P2 having been stored at 25° C. for one month were tested using a IV tester. Xe arc lamp in the IV tester was used to simulate sunlight with a known intensity and the front surface of the solar cell was irradiated to generate the IV curve. Using this curve, various parameters common to this measurement method which provide for electrical performance comparison were determined, including Eta (efficiency of solar cell), short circuit current density (Isc), open circuit voltage (Voc), Fill Factor (FF), series resistance (Rs), series resistance under three standard lighting intensities (Rs3), and front grid resistance (GRFr3 or Rfront). All data are shown in Table 4. The solar cell prepared with the Control paste (commercial SOL 9621) in Table 2 is used as the control.

TABLE 4

| Cell | Eta | Isc | Jsc | Voc | FF | Rs | Rs3 | Rsh | GRFr3 | J01 | J02 |
|---|---|---|---|---|---|---|---|---|---|---|---|
| Control | 19.08 | 9.033 | 37.81 | 0.6374 | 79.18 | 0.0043 | 0.0031 | 628.7 | 45.9 | 0.4 | 9.5 |
| P1 | 19.04 | 9.023 | 37.77 | 0.6396 | 78.83 | 0.0046 | 0.0034 | 803.2 | 58.9 | 0.4 | 9.9 |
| P2 | 18.96 | 9.002 | 37.68 | 0.6390 | 78.75 | 0.0046 | 0.0033 | 527.5 | 60.3 | 0.4 | 9.4 |

As shown through the results listed in Table 4, the experimental pastes P1 and P2 exhibited acceptable series resistance (Rs), front grid resistance (GRFr3), conductivity and overall solar cell efficiency (Eta). Furthermore, the electrical performance of the water-based paste is at least comparable to the traditional organic-based paste while being environmentally friendly.

These and other advantages of the invention will be apparent to those skilled in the art from the foregoing specification. Accordingly, it will be recognized by those skilled in the art that changes or modifications may be made to the above described embodiments without departing from the broad inventive concepts of the invention. Specific dimensions of any particular embodiment are described for illustration purposes only. It should therefore be understood that this invention is not limited to the particular embodiments described herein, but is intended to include all changes and modifications that are within the scope and spirit of the invention.

What is claimed:

1. A water-based vehicle for use in an electroconductive paste comprising:
a binder;
a stabilizer; and
water.

2. The water-based vehicle vehicle of claim 1, wherein the water is above 50 wt % of the water-based vehicle.

3. The water-based vehicle of claim 1, wherein the binder is at least one of polyvinylpyrrolidone, polyvinyl alcohol, and polyethelene glycol.

4. The water-based vehicle of claim 1, further wherein the binder is from about 5 to about 30 wt % of the water-based vehicle.

5. The water-based vehicle of claim 1, wherein the stabilizer is ethylene glycol, propylene glycol, 1,4-butane diol, or diethylene glycol.

6. The water-based vehicle of claim 1, wherein the stabilizer is about 3-30 wt % of the water-based vehicle.

7. The water-based vehicle of claim 1, wherein the stabilizer is ethylene glycol.

8. The water-based vehicle of claim 7, wherein the ethylene glycol is 5-10 wt % of the water-based vehicle.

9. The water-based vehicle of claim 1, further comprising a thixatropic agent.

10. The water-based vehicle of claim 1, wherein a weight ratio of water to ethylene glycol is from 7 to 15.

11. An electroconductive paste for use in solar cell technology comprising:
a metallic particle;
a glass frit; and
a water-based vehicle of claim 1,
wherein the water-based vehicle is about 1-20 wt % of the electroconductive paste.

12. The electroconductive paste of claim 11, wherein the metallic particle is about 60-90 wt % of the electroconductive paste, further wherein the metallic particles are at least one of silver, gold, copper, and nickel.

13. The electroconductive paste of claim 11, wherein the glass frit is about 1-10 wt % of the electroconductive paste.

14. The electroconductive paste of claim 11, further comprising a thixatropic agent.

15. A solar cell produced by applying an electroconductive paste of claim 11 to a silicon wafer, and firing the silicon wafer.
